(19) United States
(12) Patent Application Publication
(10) Pub. No.: US 2015/0351702 A1
(43) Pub. Date: Dec. 10, 2015

(54) DEVICE FOR ASSESSING DROWSINESS LEVEL

(71) Applicant: HITACHI, LTD., Chiyoda-ku, Tokyo (JP)

(72) Inventors: MIYUKI KONO, Tokyo (JP); Kazuyuki SHIMADA, Tokyo (JP)

(73) Assignee: HITACHI, LTD., Tokyo (JP)

(21) Appl. No.: 14/758,916

(22) PCT Filed: Mar. 15, 2013
(86) PCT No.: PCT/JP2013/057340

§ 371 Date: Jul. 1, 2015

(51) Int. Cl. A61B 5/00; A61B 5/18; A61B 5/01

(52) U.S. Cl. CPC A61B 5/7278; A61B 5/01; A61B 5/746; A61B 5/6826; A61B 5/18; A61B 5/6897; A61B 5/6807; A61B 5/6806; A61B 5/741; A61B 5/742; A61B 5/7455

(57) ABSTRACT

Drowsiness of a human may be caused by an underlying disease such as sleep apnea syndrome in some cases, but not in other cases. Regardless of the cause, drowsiness occurring during driving of a vehicle or the like may in some cases lead to a serious accident involving loss of life. Accordingly, a technique is needed to analyze drowsiness level with high accuracy so as to prevent accidents and ensure safety. To solve this problem, a device for assessing drowsiness level is provided for calculating a drowsiness level of a living body based on a core body temperature and a surface temperature in a peripheral region of the living body.

### TECHNICAL FIELD

The present invention relates to a technique to detect drowsiness of a human.

### BACKGROUND ART

JP 2011-123653 A (PTL 1) discloses a background art of this technical field. As stated in this publication, “. . . assesses the driver's arousal level based on data obtained from the temperature of the facial skin, the temperature of the finger skin and the pulse rate, each being measured, and on each assessing threshold value” (refer to Solution to Problem).

### CITATION LIST

### Patent Literature

PTL 1: JP 2011-123653 A

### SUMMARY OF INVENTION

### Technical Problem

Drowsiness of a human may be caused by an underlying disease such as sleep apnea syndrome in some cases, but not in other cases. Regardless of the cause, drowsiness occurring during driving of a vehicle or the like may in some cases lead to a serious accident involving loss of life. Furthermore, drowsiness has already led to heavy economic losses. Therefore, there is a need for a technique to detect drowsiness (before falling asleep) with such a high accuracy as to classify the drowsiness into levels, and prevent accidents and ensure safety by taking measures corresponding to each level.

The known technique in the above publication may be able to detect a fixed level of drowsiness to assess the drowsiness based on the preset assessing threshold; however, it is difficult to detect the drowsiness with high accuracy.

### Solution to Problem

To solve the above-described problem, configurations as described in CLAIMS are adopted, for example.

The present application includes a plurality of means for solving the above-described problem, and provides an exemplary means for assessing a drowsiness level of a living body by calculating the drowsiness level based on a core body temperature and a surface temperature in a peripheral region of the living body.

### Advantageous Effects of Invention

Detecting drowsiness with high accuracy makes it possible to prevent accidents caused by drowsiness. As a result, safety for humans and society can be achieved and economic loss can be reduced.

### BRIEF DESCRIPTION OF DRAWINGS

FIG. 1 is an exemplary schematic diagram of an in-vehicle device for assessing drowsiness level.

FIG. 2 is an exemplary overall configuration of a device for assessing drowsiness level.

FIG. 3 is an exemplary flowchart illustrating processing for assessing drowsiness level.

FIG. 4 is exemplary temperature measurement data during an occurrence of drowsiness.

FIG. 5 is exemplary temperature measurement data during the state of feeling absent-minded without any occurrence of drowsiness.

FIG. 6 is exemplary temperature measurement data during the state of feeling awake.

FIG. 7 is an exemplary drowsiness level assessing table.

FIG. 8 is an exemplary schematic diagram of a device for assessing drowsiness level for a terminal operator.

### DESCRIPTION OF EMBODIMENTS

Embodiments will be described below with reference to the drawings.

### First Embodiment

In the present embodiment, an exemplary in-vehicle drowsiness detection apparatus (100) will be described.

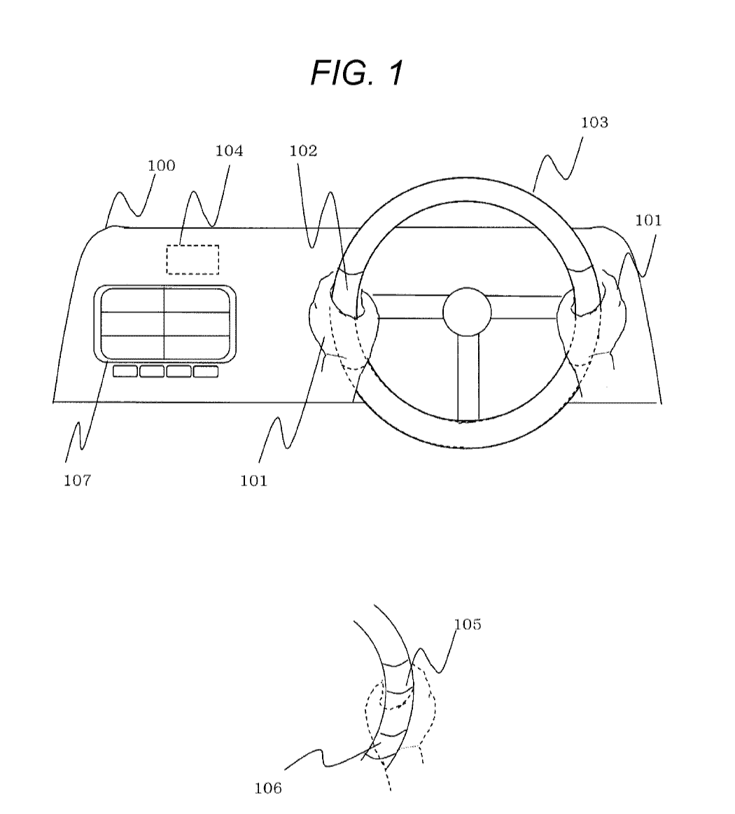

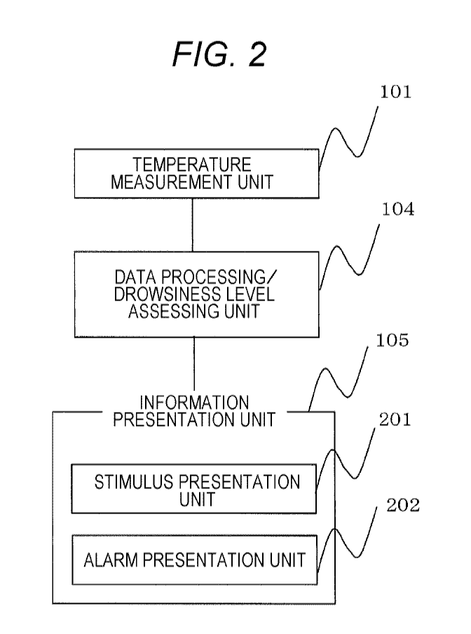

FIG. 1 is an exemplary schematic diagram of an in-vehicle drowsiness detection apparatus according to the present embodiment. Electrical wiring is omitted in FIG. 1. FIG. 2 illustrates an exemplary drowsiness detection apparatus.

A temperature measurement unit (102) for measuring the temperature of a human hand (101) may be attached to a surface or an inner portion of a steering wheel (103), or to the human hand. Alternatively, a non-contact temperature sensor may be installed in a vehicle or the inner portion of the steering wheel, or mounted on the shift lever. Therefore, techniques in the embodiment are merely exemplary and non-limiting. In use of the sensor attached to the human hand (101), data may be transmitted to a data processing/drowsiness level assessing unit (104) using a radio wave or the like, other than using a cable.

The temperature measurement unit (102) may be separated into a fingertip temperature measurement unit (105) and a palm temperature measurement unit (106). Another possible technique is to provide a temperature sensor on one surface; the sensor portion to be used for the measurement is determined using a pressure sensor to detect a hand touch. According to the drowsiness level calculated by the data processing/drowsiness level assessing unit (104), an information presentation unit (107) is functionally configured and includes a stimulus presentation unit (201) and an alarm presentation unit (202). The information presentation unit (107) presents a stimulus to alert a driver, or presents an alarm to the driver and outside the vehicle. A technique using the information presentation unit (107) in conjunction with a vehicle navigation system is effective, but non-limiting. The stimulus and alarm as described above may be presented as voice, or visual information such as a character, an image, and a video presented on a display. As a stimulus, cool air can be blown in conjunction with an air conditioner. Other techniques include generating fragrance. Examples of fragrances known to have alertness effects include rosemary, peppermint, and tea tree. An effective technique to use essential oils having alertness effects may be to provide essential oils in a bottle or provide fragrance components such as essential oil on a tray, to be placed at an air conditioning outlet in advance. When a driver needs to be alerted, air from the air conditioner containing fragrance components may be blown with this technique. An alarm to the outside of a vehicle may be implemented by reporting to an external institution on a radio wave or the like, by flashing a hazard lamp, or by using a message board such as an electronic bulletin board installed inside or outside the vehicle. An alarm can be displayed in conjunction with a destination screen or a passenger status screen when the vehicle is a bus.

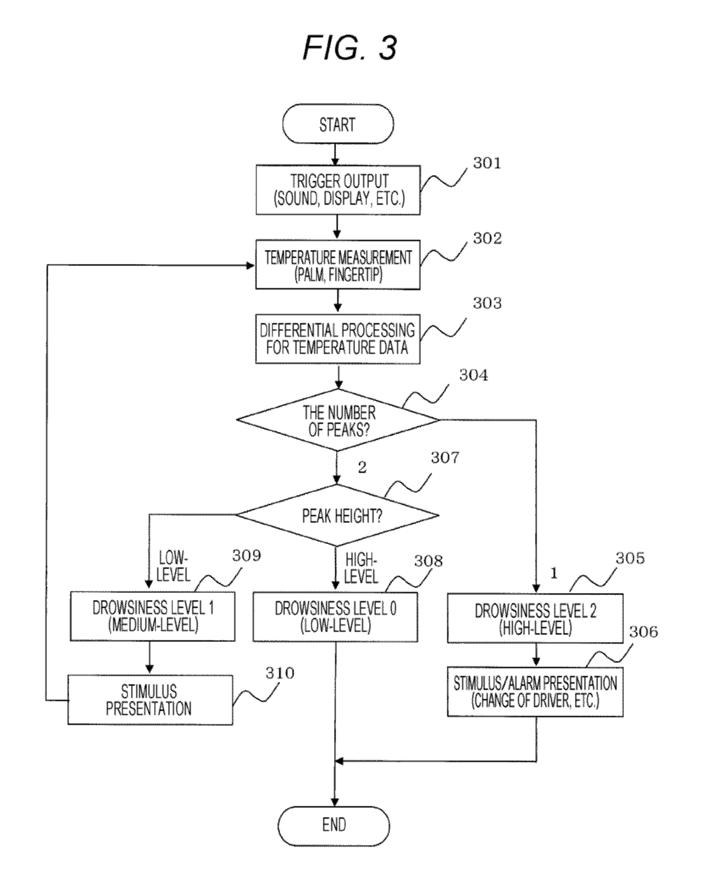

An exemplary method of detecting drowsiness in a vehicle will be described specifically below by using a flowchart in FIG. 3. A trigger (301) to start a temperature measurement (302) may be emitted as a sound generated from a speaker, an image and a character displayed with voice on the information presentation unit or the like. Effective temperature measurement regions of a body include a palm, which possibly and relatively reflects a core body temperature in a peripheral region, and a fingertip having a surface temperature of a peripheral region tip. These are exemplary and non-limiting. A temperature is measured at a preset time interval (0.1 sec. or 1 sec., for example) over a period of time, and a time-differential value for the specific time is calculated (303). The differential value is calculated as the average rate of changes at a preset time interval (1 sec. or 10 sec., for example). By observing a temporal change of the time-differential value, the presence or absence of a peak can be detected to calculate a drowsiness level. The use of the above two techniques makes it possible to analyze the drowsiness level with higher accuracy than conventional techniques. In order to assess that a certain portion of a curve has a peak, it may be sufficient to set a threshold or use a margin from adjacent values of a point, as appropriate.

When peaks are lost in a palm and one peak is detected in a fingertip, the assessment is drowsiness level 2 (high-level drowsiness), and then, the status goes on to a stimulus presentation for alertness and an alarm presentation (306) including a call for a replacement driver. When a peak is detected in both a palm and a fingertip, peak heights (absolute values) and/or time intervals of the peaks can be used for the assessment. In the assessment using peak heights, the peak height lower than a preset threshold is assessed as drowsiness level 1 (medium-level drowsiness). In this case, stimulus presentation (310) is performed while a processing flow on and after the temperature measurement is repeated to confirm the effectiveness of the stimulus presented. The difference in the forms of the peaks can be used in this manner to analyze the drowsiness level with higher accuracy.

When time intervals are used instead of the peak heights for assessment, it is assessed as follows: the shorter the time intervals of the peaks, the lower the drowsiness level; the longer the time intervals of the peaks, the higher the drowsiness level. Thresholds are also preset in this case.

Thresholds for the peak heights and time intervals are not for everybody, and can be set individually using own data.

If no peaks are detected in palm and fingertip, the driver has possibly fallen asleep already. An object of the present invention is to detect drowsiness before reaching this state. If this state is detected, an alarm using a hazard lamp, voice, a message board or the like is emitted preferentially to the outside of the vehicle, and a stimulus is provided to the driver for alertness.

A trigger (301) for starting a measurement can be programmed in advance and emitted during driving at a specified time interval. Alternatively, a trigger may be emitted by, for example, using a timing of operating a brake or a shift lever, while stopping at an intersection or at a timing of starting a vehicle upon a traffic signal change.

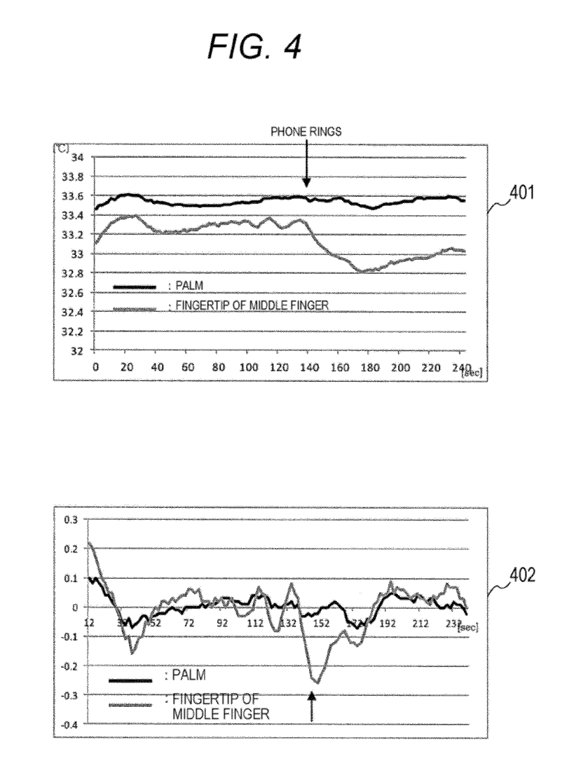

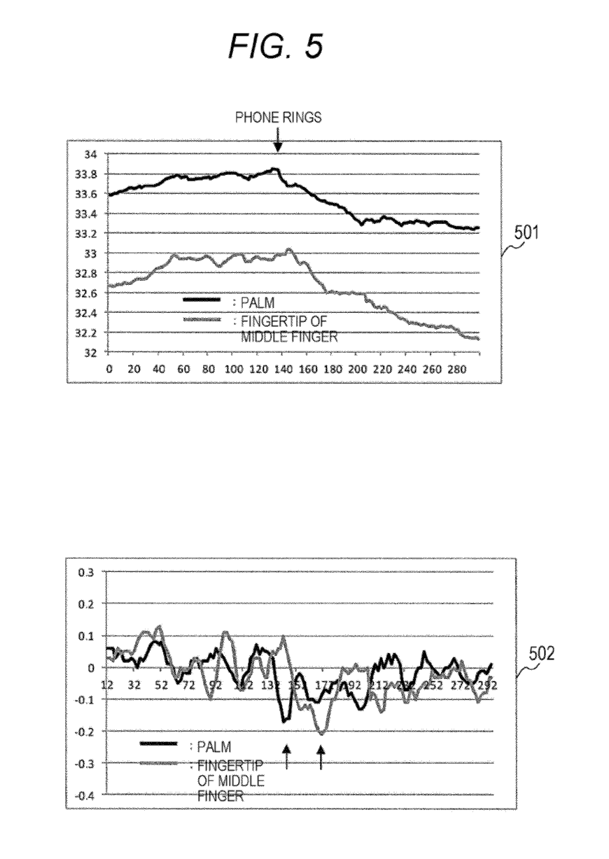

Exemplary data of measured temperature and exemplary processing data are shown in FIGS. 4 to 6. FIG. 4 is exemplary data in a case where a human is feeling drowsiness, and continuing PC operations while hearing the phone ring. According to the temperature data of a palm and a fingertip when the human is feeling drowsiness (401), the temperature of the fingertip begins to fall immediately after the human has heard the phone ring, but the temperature of the palm has not changed much. According to the time-differential data of the temperature changes when the human is feeling drowsiness (402), a differential peak can be observed in the fingertip data, while no great differential peak waveform is observed in the palm data. Here, the differential data represent plotted results of average variation rates measured at 12-second intervals. FIG. 5 is exemplary data for a case where a human is absent-minded, and starts conversation on a phone. According to the temperature data of a palm and a fingertip when the human is feeling no drowsiness but is absent-minded (501), the temperature of the palm falls immediately after the human has heard the phone ring, and then the temperature of the fingertip falls. According to the time-differential data of temperature changes when the human is feeling no drowsiness but is absent-minded (502), a differential peak of temperature changes of a palm appears, and then, a differential peak of temperature changes of a fingertip appears.

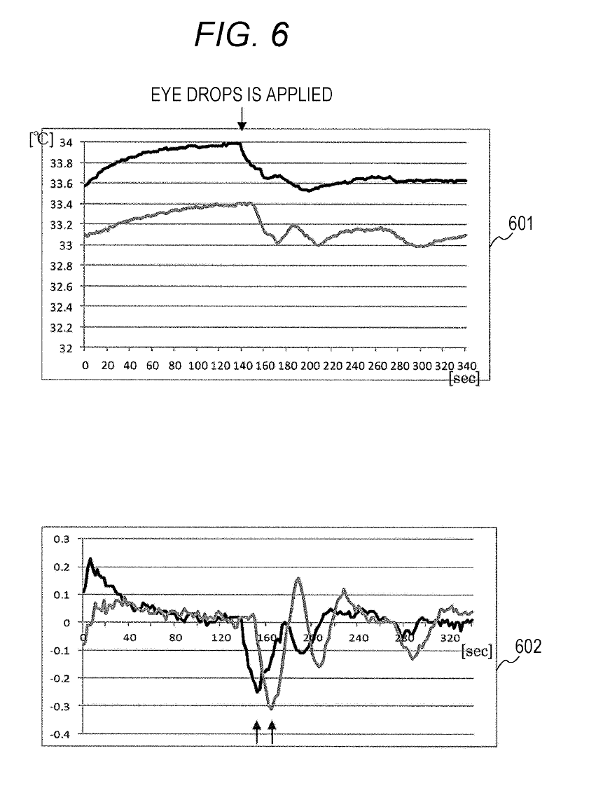

FIG. 6 is exemplary data for a case where a human has used eye drops while feeling no drowsiness. According to the temperature data of a palm and a fingertip when the human is feeling awake (601), the temperature of the palm falls immediately after a stimulus (eye drops) is applied, and then the temperature of the fingertip falls. According to the time-differential data of temperature changes when the human is feeling awake (602), a differential peak of temperature changes of the palm appears, and then, a differential peak of temperature changes of the fingertip appears. Note that the interval between these two differential peaks is shorter, and the forms of the two peaks are sharper, than the results shown in the time-differential data of temperature changes when the human is feeling no drowsiness but is absent-minded (502).

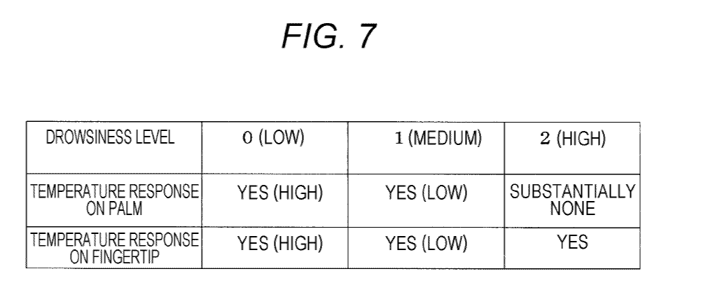

FIG. 7 is an exemplary drowsiness level assessing table. When the interval between two peaks is shorter than a preset threshold, or the forms of the two peaks are sharp, it is defined as having a high temperature response. When the temperature responses to a given stimulus are high both on a palm and a fingertip, it can be assessed that the drowsiness level is low. When the temperature responses on both the palm and the fingertip are weak, it can be assessed that the drowsiness level is medium. When there is substantially no temperature response on the palm, it can be assessed that the drowsiness level is high even if the temperature response on the fingertip is slightly detected. The drowsiness level assessing table is non-limiting. It is possible to include cases such as when the temperature response on the palm is high and the temperature response on the fingertip is low.

To assess the drowsiness level, it is possible not only to use differential data but also to detect a difference between temporally successive data so as to assess whether the temperature is on the rise. A second point is measured after a predetermined time of 1 sec., for example, has passed since a first point is measured. A difference between the two measurement values is then calculated. Furthermore, after a predetermined period of time, a third point is measured. Then, a difference between the measurement value of the second point and the measurement value of the third point is calculated. This operation is continued and when the difference remains a positive value continually, the temperature is assessed to be on the rise. In this manner, the drowsiness level can be analyzed with high accuracy.

### Second Embodiment

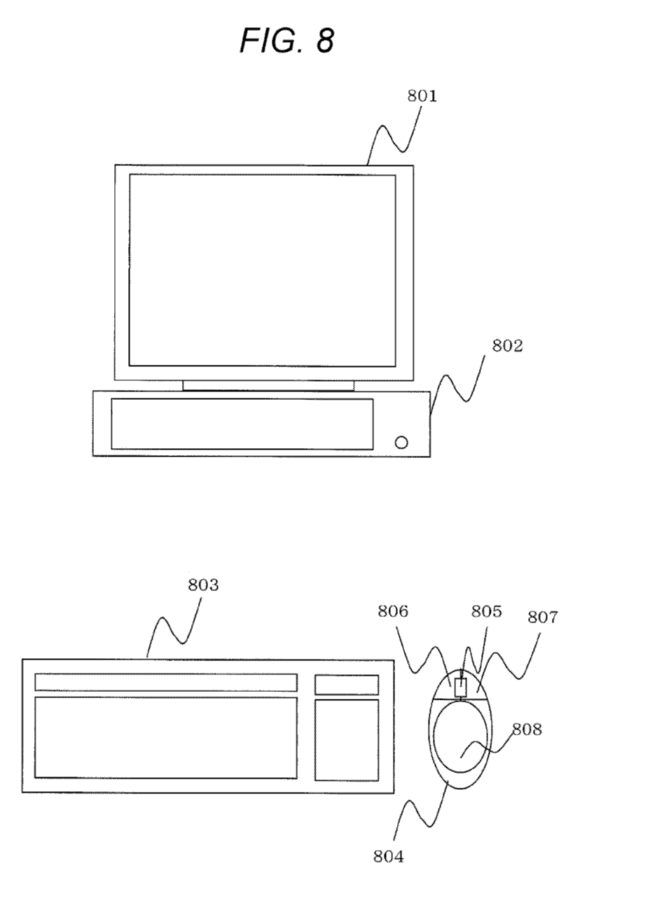

In the present embodiment, another example of a device for assessing drowsiness level will be described. Here, the apparatus may be used not only in a vehicle but also by a system terminal operator, a PC operator or the like. FIG. 8 is an exemplary schematic diagram of a device for assessing drowsiness level 800 for a terminal operator in the second embodiment.

Here, description will be omitted for the portions with the same reference signs and the same functions as in the already-described configuration of the in-vehicle drowsiness detection apparatus 100 in FIG. 1.

Electrical wiring among a monitor (801), a computer (802), a keyboard (803), and a mouse (804) is omitted in FIG. 8. The temperature of a human hand may be measured using the keyboard (801) and the mouse (802), but the measurement method is non-limiting. For the measurement using the mouse (804), a sensor for a fingertip temperature measurement is attached to a wheel button (305), a left button (306), and a right button (307). Functions of the left/right buttons for a left-handed operator are reversed from the functions of the left/right buttons for a right-handed operator.

A palm temperature measurement unit (106) is attached to a portion (808) to be covered with a palm when the mouse is operated with a human hand. There are other techniques such as attaching a surface thermometer to the mouse surface, or attaching a non-contact temperature sensor to the inner structure of the mouse. The drowsiness level can be analyzed with high accuracy by using the above techniques without much costs, and without interrupting normal computer operations.

Procedures for a temperature measurement and a drowsiness level assessment are similar to the procedures in the first embodiment. A monitor (801) can be used as an information presentation unit (107). Stimulus presentation (310) and stimulus/alarm presentation (306) can be implemented by using the monitor (801), voice with a speaker, or the like. Other techniques include providing a vibration function to the mouse (804) or the keyboard (803) and presenting a stimulus to an operator using the vibration. In presenting the above using the monitor (301), a drowsiness detection program or the like is started in advance on a computer terminal (302) or on a system to which the terminal is coupled. In order to prevent an accident caused by, for example, an erroneous operation on the terminal, it is effective to present an alarm, together with information on a problematic terminal, to a system administrator, a labor manager, or the like.

### Third Embodiment

In the present embodiment, an exemplary device for assessing drowsiness level will be described in which a temperature measurement unit (102) is worn by a human and not attached to an apparatus operated by the human, so that drowsiness can be detected substantially wherever the human is. This is effective, for example, for detecting drowsiness of a surveillance staff standing on a ship.

If a portion to be measured is a foot, a technique to use a sock is effective, and if the portion is a hand, a technique to use a glove is effective, but non-limiting. For a sock, effective techniques include attaching a temperature measurement sensor to fiber portions that come in contact with a sole and a fingertip. For a glove, effective techniques include attaching a temperature measurement sensor to fiber portions that come in contact with a palm and a fingertip. Furthermore, there are other effective techniques to incorporate a sensor formed of a material such as carbon nanotubes, which may be fabricated into the fiber. It is also possible to place carbon nanotubes between the temperature measurement sensor and a body surface.

An electrical circuit in a detection system beyond the sensor may be provided outside the glove. Alternatively, a wireless function can be provided in the sensor so that the data can be transmitted wirelessly, but these methods are non-limiting.

Attaching a temperature sensor to footwear such as thong sandals may be effective, other than attaching the sensor to the above-described sock or glove including the glove for drive. In this manner, drowsiness level can be analyzed with high accuracy substantially whenever the human is.

### REFERENCE SIGNS LIST

100 in-vehicle drowsiness detection apparatus
101 human hand
102 temperature measurement unit
103 steering wheel
104 data processing/drowsiness level assessing unit
105 fingertip temperature measurement unit
106 palm temperature measurement unit
107 information presentation unit
201 stimulus presentation unit
202 alarm presentation unit
301 trigger
302 temperature measurement
303 differential processing for temperature data
304 assessment of the number of peaks
305 assessment of drowsiness level 2 (high-level drowsiness)
306 stimulus/alarm presentation
307 assessment of peak height
308 assessment of drowsiness level 0 (low-level drowsiness)
309 assessment of drowsiness level 1 (medium-level drowsiness)
310 stimulus presentation
401 temperature data of palm and fingertip when human is feeling drowsiness
402 time-differential data of temperature changes when human is feeling drowsiness
501 temperature data of palm and fingertip when human is feeling no drowsiness but is absent-minded
502 time-differential data of temperature changes when human is feeling no drowsiness but is absent-minded
601 temperature data of palm and fingertip when human is feeling awake
602 time-differential data of temperature changes when human is feeling awake
800 device for assessing drowsiness level for terminal operator
801 monitor
802 computer terminal
803 keyboard
804 mouse
805 scroll button
806 left button
807 right button
808 portion covered with palm when mouse is operated with human hand

1. A drowsiness level analyzing apparatus comprising:
a temperature acquisition unit configured to acquire a core body temperature and a surface temperature in a peripheral region of a living body; and
a drowsiness level assessing unit configured to calculate a drowsiness level of the living body based on the acquired core body temperature and surface temperature.

2. The drowsiness level analyzing apparatus according to claim 1, wherein
the drowsiness level assessing unit is configured to acquire a time-differential value of the core body temperature and a time-differential value of the surface temperature separately, and calculate the drowsiness level based on the time-differential value of the core body temperature and the time-differential value of the surface temperature.

3. The drowsiness level analyzing apparatus according to claim 1, further comprising:
an information presentation unit configured to present information based on the drowsiness level.

4. The drowsiness level analyzing apparatus according to claim 3, wherein
the information presentation unit further includes a stimulus presentation unit configured to provide a stimulus to the living body based on the drowsiness level.

5. The drowsiness level analyzing apparatus according to claim 1, wherein the core body temperature is a temperature of a palm, and the surface temperature is a temperature of a fingertip.

6. The drowsiness level analyzing apparatus according to claim 5, wherein
the temperature acquisition unit is a steering wheel, and includes:
a steering wheel surface temperature acquisition unit attached to a surface or an inner portion of the steering wheel and configured to acquire the core body temperature; and
a steering wheel finger region temperature acquisition unit attached onto the steering wheel and configured to acquire the surface temperature.

7. The drowsiness level analyzing apparatus according to claim 5, wherein
the temperature acquisition unit is a mouse for computer operation, and includes:
a mouse surface temperature acquisition unit attached to a surface of the mouse for computer operation and configured to acquire the core body temperature; and
a mouse-button temperature acquisition unit provided on a button attached to the mouse for computer operation and configured to acquire the surface temperature.

8. The drowsiness level analyzing apparatus according to claim 5, wherein
the temperature acquisition unit is a sock, and includes:
a sole temperature acquisition unit attached to a fiber in a sole region of the sock and configured to acquire the core body temperature; and
a toe temperature acquisition unit attached to a fiber in a fingertip region of the sock and configured to acquire the surface temperature.

9. The drowsiness level analyzing apparatus according to claim 5, wherein
the temperature acquisition unit is a glove, and includes:
a palm temperature acquisition unit attached to a fiber in a palm region of the glove and configured to acquire the core body temperature; and
a finger temperature acquisition unit attached to a fiber in a fingertip region of the glove and configured to acquire the surface temperature.
